(19) United States
(12) Patent Application Publication
(10) Pub. No.: US 2005/0192337 A1
(43) Pub. Date: Sep. 1, 2005

(54) Processes and polymorphs of diaryl-indolone galr3 antagonists

(76) Inventors: Vrej Jubian, Mahwah, NJ (US); John M. Wetzel, Fair Lawn, NJ (US); Michael O'Neill, Concord, OH (US)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(21) Appl. No.: 10/637,214

(22) Filed: Aug. 7, 2003

Related U.S. Application Data
(60) Provisional application No. 60/401,887, filed on Aug. 7, 2002.

(51) Int. Cl. A61K 31/404; C07D 209/36

(52) U.S. Cl. 514/418; 548/484

(57) ABSTRACT

This invention relates to crystalline forms, as well as an amorphous form of Compound I [1-phenyl-3-[[3-(trifluoromethyl)phenyl]imino]-1H-indol-2-one], the processes for their preparation, compositions containing the same and the therapeutic use of such compositions. This invention further relates to a process for preparing Compound I.

This application claims the benefit of U.S. Provisional Application No. 60/401,887, filed on Aug. 7, 2002, the contents of which is hereby incorporated by reference into this application.

### BACKGROUND OF THE INVENTION

Throughout this application, various publications are referenced by providing full citations for these references. The disclosures of these publications in their entireties are hereby incorporated by reference into this application to describe more fully the art to which this invention pertains.

This present invention relates to a novel and more efficient process of manufacturing Compound I, which is known by the chemical name 1-phenyl-3-[[3-(trifluoromethyl)phenyl]imino]-1H-indol-2-one.

This present invention further relates to novel crystalline forms of Compound I useful as pharmaceutical agents, to methods of production and isolation of such crystalline forms of Compound I, to pharmaceutical compositions containing such crystalline forms, and to methods of treating human disorders using such crystalline forms.

Compound I (1-phenyl-3-[[3-(trifluoromethyl)phenyl]imino]-1H-indol-2-one) is a GalR3 antagonist that is useful for the treatment of depression and/or anxiety (Blackburn, T. et al., U.S. Ser. No. 10/066,175, hereby incorporated by reference in its entirety).

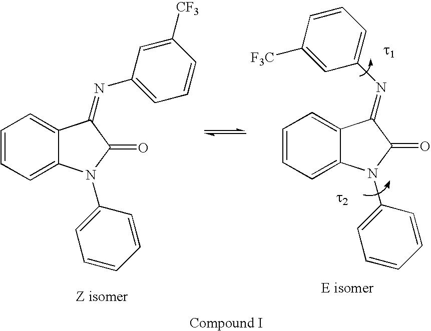

At room temperature in solution, Compound I exists as a mixture of rapidly inter-converting E and Z imine isomers, as indicated in the above drawing.

Compound I can be purchased in research quantities (milligrams) from Bionet Research Ltd., 3 Highfield Industrial Estate, Camelford, Cornwall PL32 9QZ, UK. However, this vendor does not offer large quantities (kilograms or tons) of Compound I, nor does it sell Compound I in a grade that is suitable for pharmaceutical use in humans.

We previously described methods for the synthesis of Compound I (Blackburn, T. et al., U.S. Ser. No. 10/066,175). However, these synthetic methods may not be optimal for large scale manufacturing of Compound I for pharmaceutical use.

Compound I is a derivative of 1-phenylisatin. We reported that Compound I can be prepared by imine formation between 1-phenylisatin and 3-(trifluoromethyl)aniline. There are a variety of methods to prepare 1-phenylisatin. The most direct method involves N-arylation of isatin using metal catalysts and a coupling reagent such as a triarylbismuth (D. M. T. Chan, Tetrahdron Lett., 1996, 37, 9013-9016), an aryl halide (G. M. Coppola, J. Heterocyclic Chem., 1987, 24, 1249-1251), or a phenylboronic acid (D. M. T. Chan, K. L. Monaco, R. Wang and M. P. Winters, Tetrahedron Lett., 1998, 39, 2933-2936). These methods use stoichiometric quantities of catalysts, provide poor yields or incomplete reactions, and require elaborate work-up conditions and/or undesirable chromatographic purification techniques to remove starting materials and side-products in order to isolate the desired product. Moreover, these methods lack “atom-economy”, defined as (Molecular Weight of Desired Product/Molecular Weight of All Products)×100% (B. M. Trost, Science 1991, 274, 1441), which is an important environmental and economic consideration for manufacturing process design. Recently, methods have been described that use catalytic amounts of copper(II) acetate as the catalyst (P. Y. S. Lam, G. Vincent, C. G. Clark, S. Deudon and P. K. Jadhav, Tetrahedron Lett. 2001, 3415-3418). Although these reactions are carried out under ambient conditions, these methods lack atom economy, requiring at least two equivalents of the phenylboronic acid coupling reagent and stoichiometric amounts of an amine oxide. Methods that use aryl halides as the coupling reagent (A. Klapars, X. Huang, and S. L. Buchwald, J. Am. Chem. Soc. 2002, 124, 7421-7428) usually involve prolonged reaction times at elevated temperatures resulting in dark reaction mixtures requiring undesirable purification methods for compound isolation. For cost-effective manufacturing, the use of any of the above approaches requires the employment of a method for isatin preparation, because this starting material can be costly and not in abundant supply. Alternatively, the intermediate, 1-phenylisatin, can also be prepared by reaction between oxalyl chloride and diphenylamine at high temperatures (W. M. Bryant et al, Synth. Commun., 1993, 23, 1617-1627) or at more ambient temperatures in the presence of a Lewis acid such as aluminum chloride or BF₃OEt₂. Thus, the existing methods for the synthesis of Compound I require two to three steps from readily available starting materials.

Thus, a need remains to develop an efficient process for the preparation of compound I starting from preferably cheap and readily available raw materials and producing the desired crystalline form in a reproducible manner. We also report the discovery of a novel chemical process that meets these objectives.

The existence of multiple crystalline and non-crystalline solid forms of a drug substance can be disadvantageous for development because a consistent grade of material must be reproducibly manufactured in order to satisfy regulatory requirements.

The synthetic methods previously described do not provide information as to the existence of crystalline forms of Compound I, nor do they provide information as to the production and isolation of various crystalline forms of Compound I. Thus, a need remains to identify different crystalline forms of Compound I from the perspective of pharmaceutical development.

We now report our discovery of three non-hygroscopic crystalline forms of Compound I, as well as an amorphous form of Compound I. Some of these forms are very stable at room temperature and highly desirable for drug development purposes.

### SUMMARY OF THE INVENTION

The present invention relates to a crystalline form of Compound I, hereby designated as Form I, which may be characterized by the X-ray powder diffraction (XRPD) pattern presented in Table 4, expressed in terms of the 2θ and relative intensities with a relative intensity of >10% as measured on a Shimadzu XRD-6000 X-ray diffractometer or equivalent using CuKα radiation.

Form I of Compound I may be further characterized as having an XRPD pattern containing one or several of the 2θ values presented in Table 4, as measured on a Shimadzu XRD-6000 X-ray diffractometer or equivalent using CuKα radiation.

Additionally or alternatively, Form I of Compound I may be characterized by the XRPD pattern similar or substantially similar to that set forth in the accompanying FIG. 1a as measured on a Shimadzu XRD-6000 X-ray diffractometer or equivalent using CuKα radiation.

Additionally, Form I of Compound I may also be characterized by differential scanning calorimetric (DSC) curves similar or substantially similar to that set forth in the accompanying FIG. 2 or FIG. 3 as measured using a TA Instruments differential scanning calorimeter 2920 or equivalent.

Furthermore, Form I of Compound I may also be characterized by its Fourier transform infrared pattern containing one or several of peaks presented in Table 14, as measured on a Magna-IR 860® (Thermo Nicolet) or equivalent.

Finally, Form I of Compound I may also be characterized by its Raman peak pattern containing one or several of peaks presented in Table 15, as measured on an FT-Raman 960 (Thermo Nicolet) spectrometer or equivalent.

The present invention further relates to one or several processes for the preparation of Form I of Compound I and/or the preparation of substantially pure Form I of Compound I.

The second aspect of the present invention relates to a second crystalline form of Compound I, hereby designated as Form II, which may be characterized by XRPD pattern presented in Table 5, expressed in terms of the 2θ and relative intensities with a relative intensity of >10% as measured on a Shimadzu XRD-6000 X-ray diffractometer or equivalent using CuKα radiation.

Form II of Compound I may be further characterized as having an XRPD pattern containing one or several of the 2θ values presented in Table 5, measured on a Shimadzu XRD-6000 X-ray diffractometer or equivalent using CuKα radiation.

Additionally or alternatively, Form II of Compound I may be characterized by an XRPD pattern similar or substantially similar to that set forth in the accompanying FIG. 1b as measured on a Shimadzu XRD-6000 X-ray diffractometer or equivalent using CuKα radiation.

Additionally, Form II of Compound I may also be characterized by the differential scanning calorimetric (DSC) curve similar or substantially similar to that set forth in the accompanying FIG. 4, as measured using a TA Instruments differential scanning calorimeter 2920 or equivalent.

Furthermore, Form II of Compound I may also be characterized by its Fourier transform infrared pattern containing one or several of peaks presented in Table 14, as measured on a Magna-IR 860® (Thermo Nicolet) or equivalent.

Finally, Form II of Compound I may also be characterized by its Raman peak pattern containing one or several of peaks presented in Table 15, as measured on an FT-Raman 960 (Thermo Nicolet) spectrometer or equivalent.

The present invention further relates to one or several processes for the preparation of Form II of Compound I and/or the preparation of substantially pure Form II of Compound I.

The third aspect of the present invention relates to a third crystalline form of Compound I, hereby designated as Form III, which may be characterized by the X-ray powder diffraction (XRPD) pattern presented in Table 6, expressed in terms of the 2θ and relative intensities with a relative intensity of >10% as measured on a Shimadzu XRD-6000 X-ray diffractometer or equivalent using CuKα radiation.

Form III of Compound I may be further characterized as having an XRPD pattern containing one or several of the 2θ values presented in Table 6, as measured on a Shimadzu XRD-6000 X-ray diffractometer or equivalent using CuKα radiation.

Additionally or alternatively, Form III of Compound I may be characterized by an XRPD pattern similar or substantially similar to that set forth in the accompanying FIG. 1c as measured on a Shimadzu XRD-6000 X-ray diffractometer or equivalent using CuKα radiation.

Additionally, Form III of Compound I may also be characterized by the differential scanning calorimetric (DSC) curve similar or substantially similar to that set forth in the accompanying FIG. 5 as measured using a TA Instruments differential scanning calorimeter 2920 or equivalent.

Furthermore, Form III of Compound I may also be characterized by its Fourier transform infrared pattern containing one or several of peaks presented in Table 14, as measured on a Magna-IR 860® (Thermo Nicolet) or equivalent.

Finally, Form III of Compound I may also be characterized by its Raman peak pattern containing one or several of peaks presented in Table 15, as measured on an FT-Raman 960 (Thermo Nicolet) spectrometer or equivalent.

The present invention further relates to one or several processes for the preparation of Form III of Compound I and/or the preparation of substantially pure Form III of Compound I.

The fourth aspect of the present invention relates to an amorphous form of Compound I, hereby designed as Amorphous Form, which may be characterized by an XRPD pattern similar or substantially similar to that set forth in the accompanying FIG. 1d as measured on a Shimadzu XRD-6000 X-ray diffractometer or equivalent using CuKα radiation.

Additionally or alternatively, Amorphous Form of Compound I may also be characterized by the differential scanning calorimetric (DSC) curve similar or substantially similar to that set forth in the accompanying FIG. 6 as measured using a TA Instruments differential scanning calorimeter 2920 or equivalent.

Additionally, Amorphous Form of Compound I may also be characterized by the differential scanning calorimetric (DSC) curves similar or substantially similar to that set forth in the accompanying FIG. 15 as measured with thermal cycling using a TA Instruments differential scanning calorimeter 2920 or equivalent.

The present invention further relates to one or several processes for the preparation of Amorphous Form of Compound I and/or the preparation of substantially pure Amorphous Form of Compound I.

One embodiment of the present invention is a composition comprising Form I, Form II, Form III, or Amorphous Form of Compound I. A further embodiment of this present invention is a composition wherein a substantial percentage of Compound I is present as Form I, Form II, Form III or Amorphous Form. Such a percentage can be in the range of at least 99.9%, 98%, 95%, 90%, 85%, 80%, 75% or 70%.

Another embodiment of the present invention is a pharmaceutical composition comprising Form I, Form II, Form III or Amorphous Form of Compound I. A further embodiment of this present invention is a pharmaceutical composition wherein a substantial percentage of Compound I is present as Form I, Form II, Form III or Amorphous Form. Such a percentage can be in the range of at least 99.9%, 98%, 95%, 90%, 85%, 80%, 75% or 70%.

Such a pharmaceutical composition is useful for treatment of a disease in a mammal suffering from such disease. One preferred embodiment is such a pharmaceutical composition useful for treatment of human depression, anxiety or other CNS disorders.

Another embodiment of the present invention is the use of a polymorphic form of Compound I for the preparation of a medicament useful for treatment of a human disease. The polymorphic form of Compound I can be Form I, Form II, Form III, or Form II. It may further exist as a mixture of two or more polymorphic forms of Compound I.

The present invention further relates to a method for treatment of a human disease, wherein the method comprises administering to a human subject suffering from such disease a therapeutically effective amount of Form I, Form II, Form III or Amorphous Form of Compound I.

In another embodiment of the method described above, the human disease is depression, anxiety or other CNS disorders.

In yet another embodiment, the method described above may further comprise admixing Form I, Form II, Form III or Amorphous Form of Compound I with a pharmaceutically acceptable carrier.

Further, the present invention provides a process of preparing Compound I, which comprises reacting diphenylamine with oxalyl chloride in a suitable solvent followed by addition of 3-(trifluoromethyl)aniline.

In one embodiment, the reaction is carried out in one pot.

In one embodiment, oxalyl chloride is replaced with an equivalent reagent from the group consisting of, but not limited to diethyloxalate or dimethyloxalate.

In one embodiment, reaction is run in a solvent selected from the group consisting of, but not limited to toluene, meta-xylene, ortho-xylene, para-xylene or an appropriate solvent.

In one embodiment, the reaction is run at a temperature range of 0° C.-200° C.

In one embodiment, the reaction is run at a temperature range of 30° C.-150° C.

In yet another embodiment, the reaction is run at a temperature range of 50° C.-135° C.

In another embodiment, the reaction is heated for a period from 1 to 48 hours.

In one embodiment, the process described above comprises the steps of combining diphenylamine with oxalyl chloride to produce 1-phenyl isatin followed by adding 3-(trifluoromethyl) aniline.

In one embodiment, the process described above further comprises steps to crystallize and isolate Compound I.

As used in this application, the term of “substantial percentage” shall have the meaning of a percentage of at least 50%. The term of “substantial purity” shall have the meaning of a purity of at least 50%.

The term of “pharmaceutical composition” shall have the meaning of a composition suitable for human pharmaceutical use.

The term of “human disease” shall have the meaning of a human disease condition. It shall include, but not be limited to, depression, anxiety and other CNS-related disorder. Depression may further comprise, but not be limited to, bipolar disorders, major depressive disorders, and dysthymic disorders. Anxiety may further include, but not be limited to, obsessive compulsive disorder (OCD), generalized anxiety disorder (GAD) and panic disorder (PD). The symptomatology and diagnostic criteria for these diseases and others are set out in the DSMIV guidelines (American Psychiatric Association (1994) Diagnostic and Statistical Manual of Mental Disorders).

The term of “CNS disorder” shall have the meaning of a human disease which relates to central nervous system.

### BRIEF DESCRIPTION OF FIGURES

The present invention is further described or illustrated by the following figures, which are herein referred to as FIGS. 1-15 or accompanying FIGS. 1-15.

FIG. 1: Representative X-Ray Powder Diffraction Patterns of Crystalline Forms I, II and III and Amorphous Form of Compound I

FIG. 2: Thermogravimetric analysis (TG) (top, 10° C./min) and differential scanning calorimetry (DSC) (bottom, 10° C./min) curves for Form I of Compound I.

FIG. 3: Differential Scanning Calorimetry Curves for Form I of Compound I.

FIG. 4: Thermogravimetric Analysis (top, 10° C./min) and Differential Scanning Calorimetry (bottom, 20° C./min) Curves for Form II of Compound I.

FIG. 5: Thermogravimetric Analysis (top, 10° C./min) and Differential Scanning Calorimetry (bottom, 20° C./min) Curves for Form III of Compound I.

FIG. 6: Thermogravimetric Analysis (top, 10° C./min) and Differential Scanning Calorimetry (bottom, 20° C./min) Curves for Amorphous Form of Compound I.

FIG. 7: ORTEP Drawing of Form I of Compound I, wherein atoms are represented by 50% probability anisotropic thermal ellipsoids.

FIG. 8: Packing diagram of Form I of Compound I (viewed down the a crystallographic axis and hydrogen atoms are omitted for clarity).

FIG. 9: Packing Diagram of Form I of Compound I (viewed down the b crystallographic axis and hydrogen atoms are omitted for clarity).

FIG. 10: Packing Diagram of Form I of Compound I (viewed down the c crystallographic axis and hydrogen atoms are omitted for clarity).

FIG. 11: ORTEP Drawing of Form II of Compound I. Atoms are represented by 50% probability anisotropic thermal ellipsoids.

FIG. 12: Packing diagram of Form II of Compound I (viewed down the a crystallographic axis and hydrogen atoms are omitted for clarity).

FIG. 13: Packing diagram of Form II of Compound I (viewed down the b crystallographic axis and hydrogen atoms are omitted for clarity).

FIG. 14: Packing diagram of Form II of Compound I (viewed down the c crystallographic axis and hydrogen atoms are omitted for clarity).

FIG. 15: Differential Scanning Calorimetry (20° C./min) Temperature Recycling Curves for Amorphous Form of Compound I.

### DETAILED DESCRIPTION OF THE INVENTION

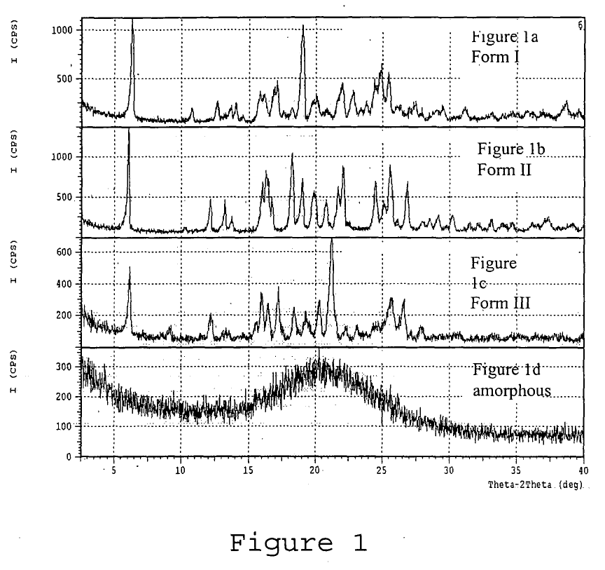

We carried out a polymorph screen in an attempt to generate as many solid forms of Compound I as possible and study their properties with the objective of determining whether crystalline or noncrystalline forms of Compound I exist and, if they exist, which form is the most desirable for pharmaceutical development. The techniques employed for the polymorph screening included fast evaporation, slow evaporation, slow cool, rotary evaporation, melt/quench, cold precipitation and vapor diffusion experiments. The solids that were generated were then analyzed by X-ray powder diffraction (XRPD). Three distinct crystalline powder patterns representing three forms were found, herein termed as Forms I, II, and III. We further identified an amorphous form of Compound I. Representative XRPD patterns of these polymorph forms are shown in FIG. 1.

Table 1 lists the polymorphic forms of Compound I that were obtained from fast evaporation, slow evaporation, slow cool, rotary evaporation, and melt/quench techniques. For these methods, a weighed sample of Compound I (usually 20-30mg) was treated with 100 μL aliquots of the test solvent. The mixture was sonicated between additions. When the solids dissolved the solution was filtered. For fast evaporation, the solution was left in an open vial under ambient conditions. For slow evaporation, the solution was left under ambient conditions in a vial that was either loosely covered with a cap or covered with aluminum foil containing pinholes. For rotary evaporation, the solvent was evaporated using a rotary evaporator. For slow cool, the sample was dissolved in a test solvent at an elevated temperature (either 45 or 60° C.). The resulting solution was rapidly filtered into a vial kept on the same hotplate. The heat source was then turned off and the hotplate and vial were allowed to cool to room temperature. The vial was then allowed to stand at ambient temperature overnight. If no solids were detected, the vial was placed in a refrigerator or freezer for overnight. For fast cool, the hot solution was placed in the freezer as soon as the solution cooled to room temperature. The melt/quench technique involved cooling a melt of compound I to room temperature.

Table 2 lists the polymorphic forms of Compound I that were obtained from cold precipitation crystallizations. A solution of Compound I was added into a vial containing a cold antisolvent (hexanes cooled in dry ice/acetone slurry or water cooled in an ice water bath).

Table 3 lists the polymorphic forms of Compound I that were obtained from vapor diffusion experiments. A saturated solution of Compound I was placed in a vial that was placed in a larger vial containing an antisolvent. The larger vial was then sealed and kept at ambient temperature.

The crystalline forms may be characterized by their x-ray powder diffraction patterns (XRPD) and/or by their differential scanning calorimetric (DSC) curves. Many samples generated in the polymorph screen exhibited preferred orientation. Preferred orientation is the tendency for crystals, usually plates or needles, to align themselves with some degree of order. Preferred orientation can affect peak intensities but not peak positions in XRPD patterns. Reduction of preferred orientation may be necessary to obtain representative XRPD patterns.

X-ray powder diffraction (XRPD) analyses were performed using a Shimadzu XRD-6000 X-ray powder diffractometer using Cu Kα radiation. The instrument was equipped with a fine focus X-ray tube. The tube voltage and amperage were set to 40 kV and 40 mA, respectively. The divergence and scattering slits were set at 1° and the receiving slit was set at 0.15 mm. Diffracted radiation was detected by a NaI scintillation detector. A theta-two theta continuous scan at 3°/min (0.4 sec/0.02° step) from 2.5 to 40° 2θ was used. A silicon standard was analyzed to check the instrument alignment. Data were collected and analyzed using XRD-6000 v. 4.1. Samples were prepared for analysis by placing them in a silicon sample holder or aluminum holder with silicon insert. X-ray powder diffraction (XRPD) analyses were also performed using an Inel XRG-3000 diffractometer equipped with a CPS (Curved Position Sensitive) detector with a 2θ range of 120°. Real time data were collected using Cu-Kα radiation starting at approximately 4° 2θ at a resolution of 0.03° 2θ. The tube voltage and amperage were set to 40 kV and 30 mA, respectively. The monochromator slit was set at 5 mm by 80 μm. The pattern is displayed from 2.5-40° 2θ. Samples were prepared for analysis by packing them into thin-walled glass capillaries. Each capillary was mounted onto a goniometer head that is motorized to permit spinning of the capillary during data acquisition. The samples were analyzed for 5 min. Instrument calibration was performed using a silicon reference standard.

Tables 4 to 6 list the 2θ values and relative intensities of all lines with a relative intensity >10% for samples of Compound I that are Forms I, II, and III respectively.

Differential scanning calorimetry (DSC) was performed using a TA Instruments differential scanning calorimeter 2920. The sample was placed into an aluminum DSC pan, and the weight accurately recorded. The pan was covered with a lid and left uncrimped. Each sample was equilibrated at 25° C. and heated under a nitrogen purge at a rate of 10 or 20° C./min, up to a final temperature of 270° C. or 350° C. Samples were also heated at 1 or 5° C./min up to 150° C.

Thermogravimetric (TG) analyses were performed using a TA Instruments 2050 or 2950 thermogravimetric analyzer. Each sample was placed in an aluminum sample pan and inserted into the TG furnace. Samples were first equilibrated at 25° C., then heated under nitrogen at a rate of 10° C./min, up to a final temperature of 350° C. Nickel and Alumel™ were used as the calibration standards.

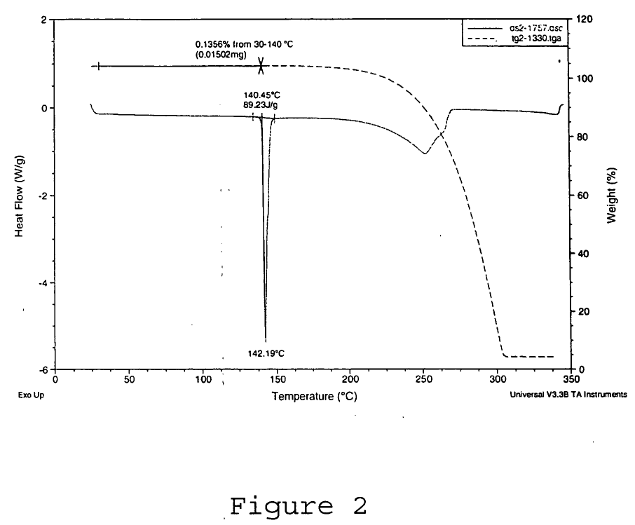

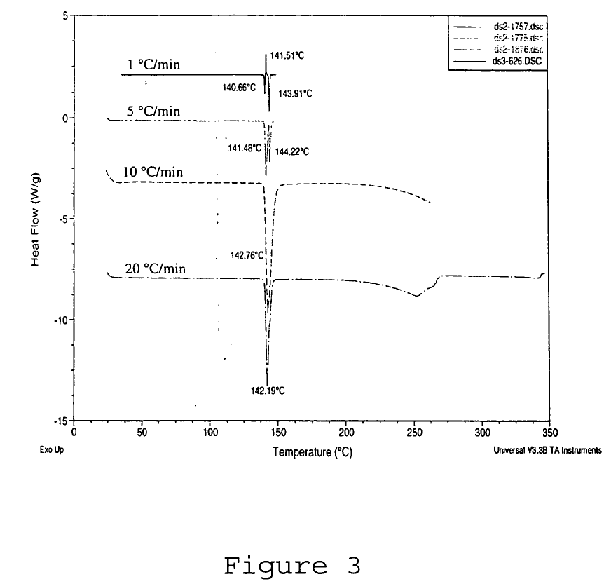

FIGS. 2,4,5, and 6 show TG analysis and DSC traces for Compound I, Forms I, II, III and amorphous form respectively. FIG. 3 shows DSC traces for Form I of Compound I at different temperature gradients.

The crystalline forms may also be characterized by infrared (IR) and/or Raman spectroscopy. Infrared spectra were acquired on a Magna-IR 860® Fourier transform infrared (FT-IR) (Thermo Nicolet). Raman spectra were acquired using an FT-Raman 960 spectrometer (Thermo Nicolet).

Table 14 provides a list of peaks from IR spectra that are unique to each of the polymorphic forms of Compound I (Form I, II, and III). Table 15 lists peaks from Raman spectra that are unique to each of the polymorphic forms of Compound I (Form I, II, and III).

Form I

Form I was crystallized from the slow cooling of ethyl acetate or toluene solutions, slow evaporation of methanol solutions, fast evaporation of diethyl ether solutions, a diethyl ether/hexanes anti-solvent crystallization, and from a vapor diffusion experiment using isopropanol and water. Based on the characterization data, Form I is a crystalline, nonhygroscopic material that melts at approximately 141° C. (DSC at 1 or 5° C./min). The thermogravimetric trace (FIG. 2), shows minimal weight loss at 140° C., which suggests that Form I is not solvated.

As described above, as measured on a Shimadzu XRD-6000 X-ray diffractometer or equivalent using CuKα radiation, Form I of Compound I displays the XRPD pattern as shown in Table 4. It should be noted that Form I of Compound I may be characterized as having one or several of the 2θ values as set forth in Table 4.

As measured on a Shimadzu XRD-6000 X-ray diffractometer or equivalent using CuKα radiation, Form I of Compound I may further be characterized by XRPD pattern similar or substantially similar to that set forth in the accompanying FIG. 1a as measured on a Shimadzu XRD-6000 X-ray diffractometer using CuKα radiation.

Alternatively, as measured using a TA Instruments differential scanning calorimeter 2920 or equivalent, Form I of Compound I may also be characterized by the differential scanning calorimetric (DSC) curve as set forth in the accompanying FIG. 2. Depending on the test conditions, the DSC curve of Form I of Compound I can be similar to or substantially similar to that as set forth in FIG. 2.

FIG. 3 shows four DSC traces for Form I measured at different rates of heating. Differences in the DSC traces were observed for Form I of Compound I depending on the temperature gradient. The trace recorded at a heating rate of 1° C./min shows two endotherms at 140.66° C. and 143.91° C. and an exotherm occurring between these temperatures at 141.51° C. This result is explained as follows: upon slowly heating Form I (1° C./min), this Form melts at approximately 141° C., then the melt crystallizes to Form II (resulting in the exotherm), which in turn melts at approximately 144° C. This result indicates that Form II is more stable than Form I at high temperatures.

Alternatively, as measured on a Magna-IR 860® Fourier transform infrared (FT-IR) (Thermo Nicolet) or equivalent, Form I of Compound I displays unique peaks as listed in Table 14. It should be noted that Form I of Compound I may be characterized as having one or several of the peaks as set forth in Table 14 (Form I).

Alternatively, as measured on an FT-Raman 960 spectrometer (Thermo Nicolet) or equivalent, Form I of Compound I displays unique peaks as listed in Table 15. It should be noted that Form I of Compound I may be characterized as having one or several of the peaks as set forth in Table 15 (Form I).

Single crystals of Form I were prepared for single crystal X-ray diffraction. A colorless plate of Compound I having approximate dimensions of 0.35×0.30×0.05 mm was mounted on a glass fiber in random orientation. Single crystals were prepared from a vapor diffusion experiment from isopropanol and water. Preliminary examination and data collection were performed with Mo K_{α} radiation (λ=0.71073 Å) on a Nonius KappaCCD diffractometer. Refinements were performed on an Alphaserver 2100 using SHELX97 [Sheldrick, G. M. SHELX97, A Program for Crystal Structure Refinement, University of Gottingen, Germany, 1997]. The crystallographic drawings were obtained using the programs ORTEP [Johnson, C. K. ORTEPII, Report ORNL-5138, Oak Ridge National Laboratory, TN, U.S.A. 1976.] and CAMERON [Watkin, D. J., Prout, C. K., Pearce, L. J. CAMERON, Chemical Crystallography Laboratory, University of Oxford, Oxford 1996.]

Cell constants and an orientation matrix for data collection were obtained from least-squares refinement using the setting angles of 20535 reflections in the range 2°<θ<25°. The refined mosaicity from DENZO/SCALEPACK [Otwinowski, Z.; Minor, W. Methods Enzymol. 1996, 276, 307.] was 0.83° indicating moderate crystal quality. The space group was determined by the program ABSEN [McArdle, P. C. J. Appl. Cryst. 1996, 29, 306]. From the systematic presence of h01 1=2n and 0k0 k=2n, and from subsequent least-squares refinement, the space group was determined to be P2₁/c (No. 14).

The data were collected to a maximum 2θ value of 51.5°, at a temperature of 150±1 K.

The monoclinic cell parameters and calculated volume are: a=7.2704(2) Å, b=16.9251(5) Å, c=27.8017(11) Å, β=92.6340(10)°, V=3417.4(3) Å³. For Z=8 and formula weight of 366.35 the calculated density is 1.42 g cm⁻³. The space group was determined to be P2₁/c (no. 14).

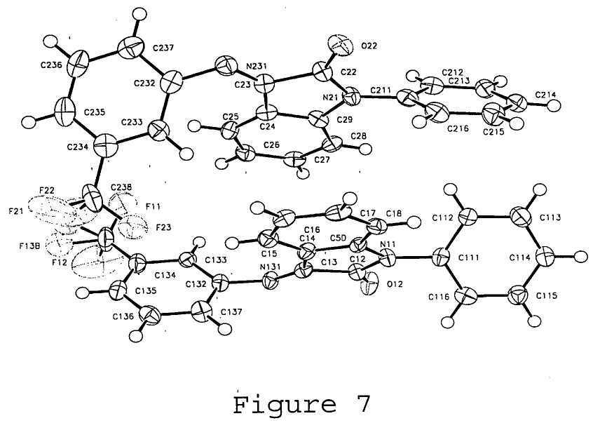

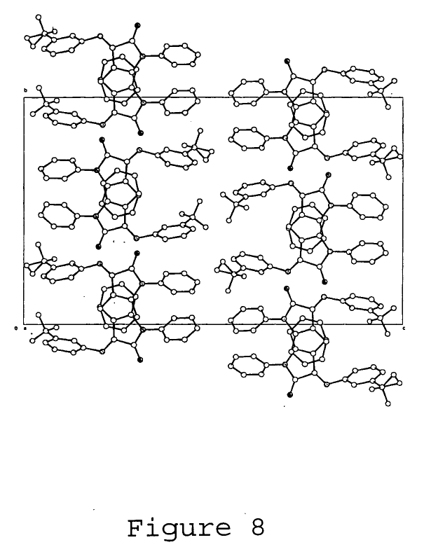

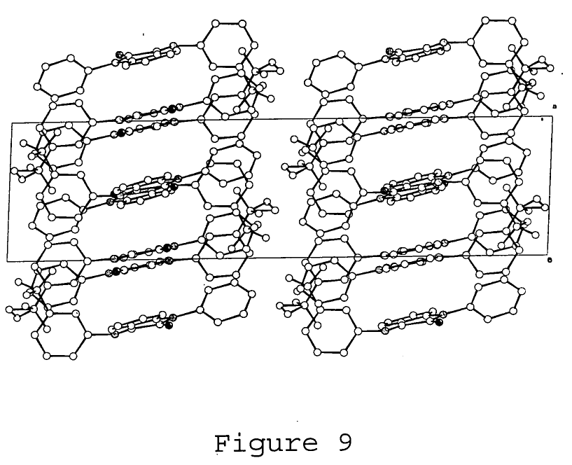

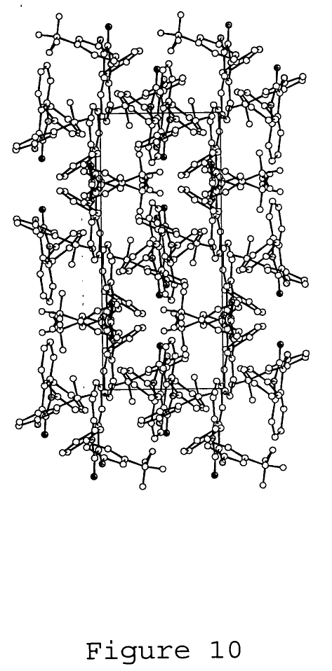

An ORTEP drawing of Compound I, Form I is shown in FIG. 7. The single crystal structure data demonstrates that the E isomer of Compound I is present in the crystals of this form. The asymmetric unit shown in FIG. 7 contains two symmetry independent molecules. These two molecules differ in the torsion angles, τ₁ and τ₂, around the C_{CF3aryl}-Nᵢₘᵢₙₒ and C_{aryl}-Nᵢₘᵢₙₒ bonds of Compound I, as listed in Table 11. Packing diagrams viewed along the a, b, and c crystallographic axis are shown in FIGS. 8, 9 and 10, respectively. Hydrogen atoms are not shown in these figures for clarity. Pairs of molecules are pi-stacked roughly along the c-axis, as shown in FIG. 9 (view along b axis). The trifluoro group on one molecule displays rotational disorder, which is observed clearly in the packing diagrams.

Form II

As described above, as measured on a Shimadzu XRD-6000 X-ray diffractometer or equivalent using CuKα radiation, Form II of Compound I displays the XRPD pattern as shown in Table 5. It should be noted that Form II of Compound I may be characterized as having one or several of the 2θ values as set forth in Table 5.

As measured on a Shimadzu XRD-6000 X-ray diffractometer or equivalent using CuKα radiation, Form II of Compound I may further be characterized by an XRPD pattern similar or substantially similar to that set forth in the accompanying FIG. 1b as measured on a Shimadzu XRD-6000 X-ray diffractometer using CuKα radiation.

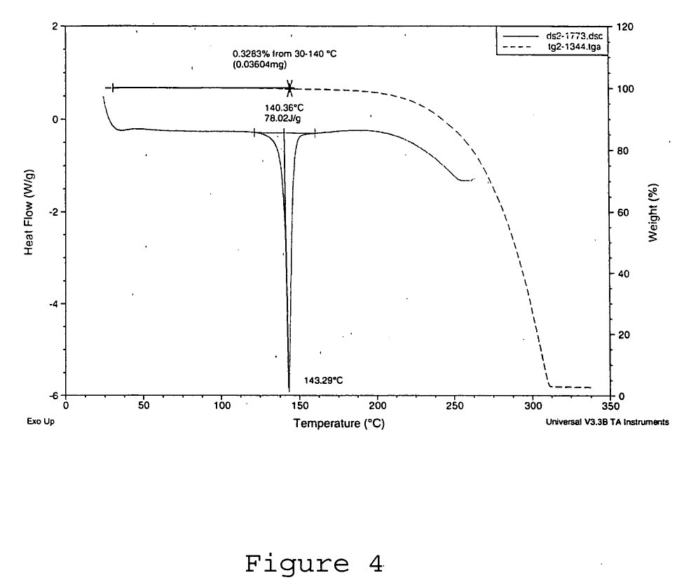

Alternatively, as measured using a TA Instruments differential scanning calorimeter 2920 or equivalent, Form II of Compound I may also be characterized by the differential scanning calorimetric (DSC) curve as set forth in the accompanying FIG. 4. Depending on the test conditions, the DSC curve of Form II of Compound I can be similar to or substantially similar to that as set forth in FIG. 4.

Tables 1-3 further reveal that Form II was the predominant product from a majority of the crystallizations attempted. Thus, Form II is the most likely form to result from any crystallization process in which no particular measures are taken to control the crystallization. The DSC shows a single endotherm at approximately 143° C. (1 or 20° C./min) which is due to the melt of the material. From the TG and DSC traces (FIG. 4), Form II is a crystalline, non-solvated material that melts at approximately 143° C. at temperature gradient of 1 or 20° C./min.

Alternatively, as measured on a Magna-IR 8600 Fourier transform infrared (FT-IR) (Thermo Nicolet) or equivalent, Form II of Compound I displays unique peaks as listed in Table 14. It should be noted that Form II of Compound I may be characterized as having one or several of the peaks as set forth in Table 14 (Form II).

Alternatively, as measured on an FT-Raman 960 spectrometer (Thermo Nicolet) or equivalent, Form II of Compound I displays unique peaks as listed in Table 15. It should be noted that Form II of Compound I may be characterized as having one or several of the peaks as set forth in Table 15 (Form II).

Single crystals of Form II were prepared for single crystal X-ray diffraction. An orange plate of Compound I having approximate dimensions of 0.38×0.33×0.13 mm was mounted on a glass fiber in random orientation.

Single crystals were prepared from the slow cooling of an ethanol solution. Preliminary examination and data collection were performed with Mo K_{α} radiation (λ=0.71073 Å) on a Nonius KappaCCD diffractometer. Refinements were performed on an Alphaserver 2100 using SHELX97 [Sheldrick, G. M. SHELX97, A Program for Crystal Structure Refinement, University of Gottingen, Germany, 1997.]. The crystallographic drawings were obtained using the programs ORTEP [Johnson, C. K. ORTEPII, Report ORNL-5138, Oak Ridge National Laboratory, TN, U.S.A. 1976] and CAMERON [Watkin, D. J. Prout, C. K., Pearce, L. J. CAMERON, Chemical Crystallography Laboratory, University of Oxford, Oxford 1996].

Cell constants and an orientation matrix for data collection were obtained from least-squares refinement using the setting angles of 13366 reflections in the range 5°<θ<27°. The refined mosaicity from DENZO/SCALEPACK [Otwinowski, Z.; Minor, W. Methods Enzymol. 1996, 276, 307] was 0.32° indicating good crystal quality. The space group was determined by the program ABSEN [McArdle, P. C. J. Appl. Cryst. 1996, 29, 306]. From the systematic presence of h01 1=2n and 0k0 k=2n and from subsequent least-squares refinement, the space group was determined to be P2₁/c (no. 14).

The data were collected to a maximum 2θ value of 55°, at a temperature of 150±1 K.

The monoclinic cell parameters and calculated volume are: a=14.7527 (2), b=16.9524 (6), c=7.0196(5) Å, β=100.8840 (19), V=1724.0 (2) Å³. For Z=4 and formula weight of 366.35 the calculated density is 1.41 g/cm⁻³. The space group was determined to be P2₁/c (No. 14).

Note that in this case, the structure has only one molecule in the asymmetric unit, which simplifies the structure relative to that seen for the Form I crystal structure.

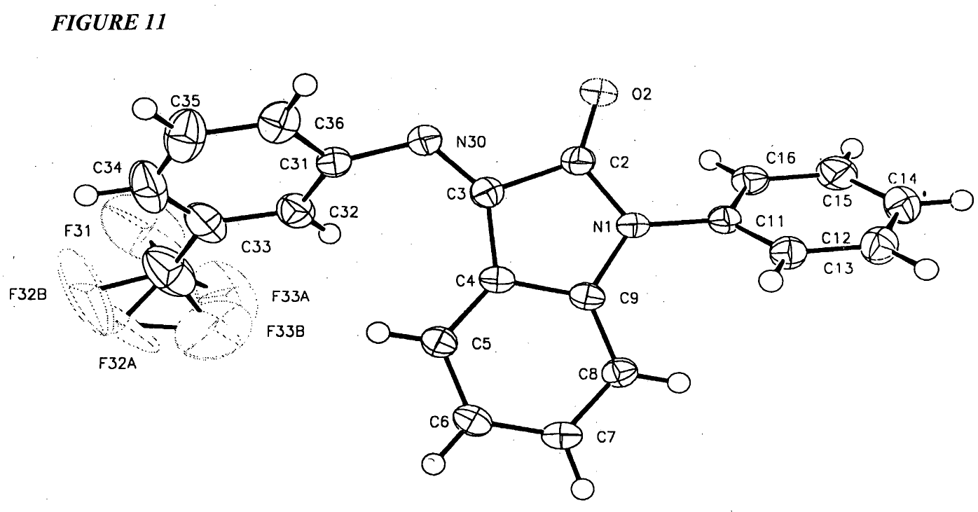

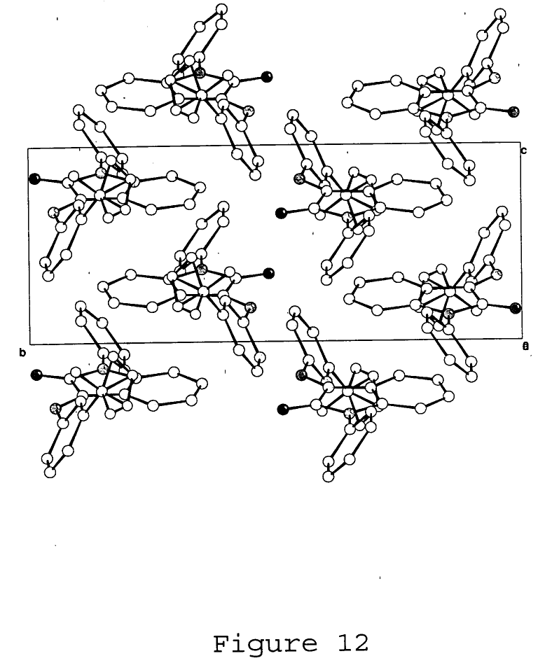

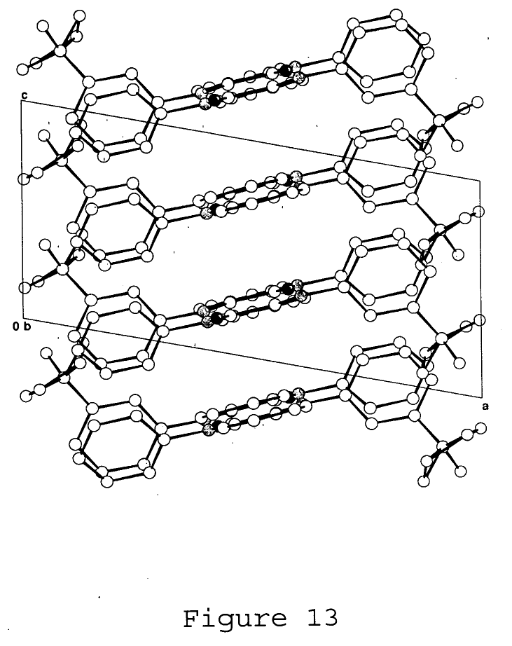

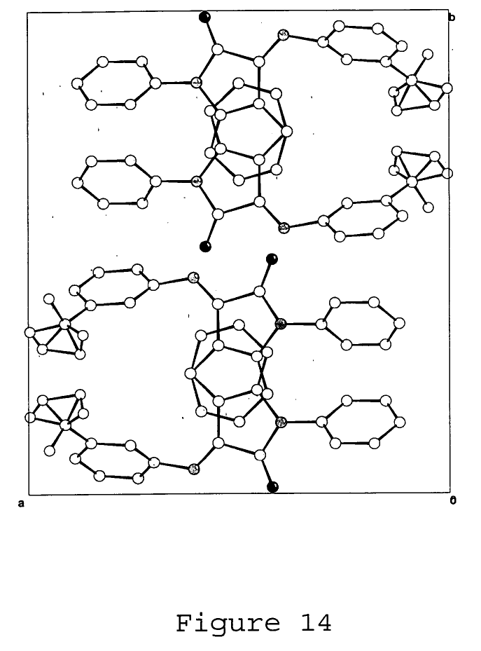

An ORTEP drawing of a molecule of Compound I in the Form II crystal is shown in FIG. 11. The single crystal structure data demonstrates that the E isomer of Compound I is present in the crystals of this form. The asymmetric unit shown in FIG. 11 contains one molecule of Compound I. Packing diagrams viewed along the a, b, and c crystallographic axis are shown in FIGS. 12, 13 and 14, respectively. Hydrogen atoms are not shown in these figures for clarity. Pairs of molecules are pi-stacked along the c-axis, as shown in FIG. 13 (b view). The disorder in the trifluoromethyl groups is also observed clearly in the packing diagrams. The molecular geometry is similar to one of the molecules in the asymmetric unit of Form I; see Table 11.

Form III

Form III was crystallized from the slow cooling of a diethyl ether solution (as a mixture with Form II), and from anti-solvent crystallization using dichloromethane and hexanes or ethyl acetate and hexanes.

As described above, as measured on a Shimadzu XRD-6000 X-ray diffractometer or equivalent using CuKα radiation, Form III of Compound I displays the XRPD pattern as shown in Table 6. It should be noted that Form III of Compound I may be characterized as having one or several of the 2θ values as set forth in Table 6.

As measured on a Shimadzu XRD-6000 X-ray diffractometer or equivalent using CuKα radiation, Form III of Compound I may further be characterized by an XRPD pattern similar or substantially similar to that set forth in the accompanying FIG. 1c as measured on a Shimadzu XRD-6000 X-ray diffractometer or equivalent using CuKα radiation.

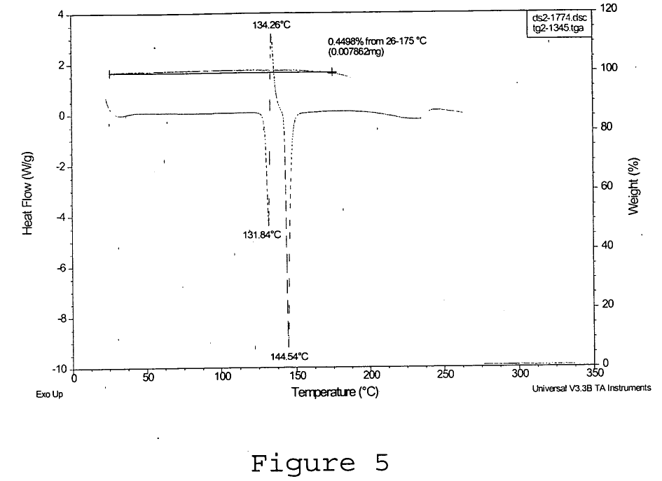

Alternatively, as measured using a TA Instruments differential scanning calorimeter 2920 or equivalent, Form III of Compound I may also be characterized by the differential scanning calorimetric (DSC) curve as set forth in the accompanying FIG. 5. Depending on the test conditions, the DSC curve of Form III of Compound I can be similar to or substantially similar to that as set forth in FIG. 5.

The TG data shows a minimal weight loss at 140° C., suggesting that the Form III material is not solvated. The DSC curve (heating rate 20° C./min) displays an endotherm at approximately 132° C. at temperature gradient of 20, immediately followed by an exotherm at approximately 134° C. and an endotherm at approximately 145° C. These events suggest that the material melted and immediately recrystallized to another phase which melts at approximately 145° C., likely Form II based on the melting point. Upon slowing the DSC heating rate (1° C./min), only one sharp endotherm is observed at approximately 144° C.; it is likely that the slow heating rate causes a solid state transformation of Form III to Form II. This data suggests that the Form III melts and subsequently recrystallizes to another form, as was seen with Form I at slow heating rates. This data also suggests that Form III is a less stable form at higher temperatures. Based on the characterization data, Form III is a crystalline, nonhygroscopic material that melts at approximately 132° C. at temperature gradient of 20° C./min and subsequently recrystallizes to another material that is likely Form II.

Alternatively, as measured on a Magna-IR 860® Fourier transform infrared (FT-IR) (Thermo Nicolet) or equivalent, Form III of Compound I displays unique peaks as listed in Table 14. It should be noted that Form III of Compound I may be characterized as having one or several of the peaks as set forth in Table 14 (Form III).

Alternatively, as measured on an FT-Raman 960 spectrometer (Thermo Nicolet) or equivalent, Form III of Compound I displays unique peaks as listed in Table 15. It should be noted that Form III of Compound I may be characterized as having one or several of the peaks as set forth in Table 15 (Form III).

Amorphous Form

Amorphous material was generated by rapidly cooling a melt of the material on a cold countertop. As measured on a Shimadzu XRD-6000 X-ray diffractometer or equivalent using CuKα radiation, Amorphous Form of Compound I may be characterized by an XRPD pattern similar or substantially similar to that set forth in the accompanying FIG. 1d as measured on a Shimadzu XRD-6000 X-ray diffractometer using CuKα radiation.

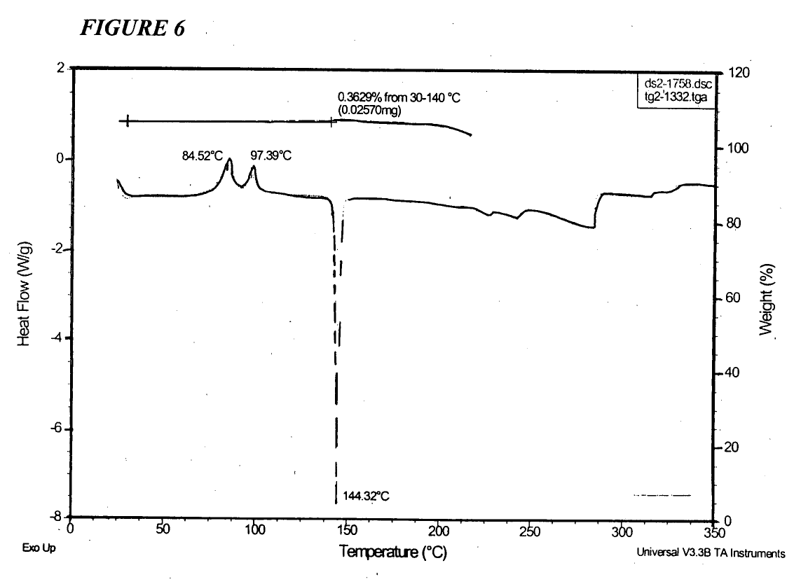

Alternatively, as measured using a TA Instruments differential scanning calorimeter 2920 or equivalent, Amorphous Form of Compound I may also be characterized by the differential scanning calorimetric (DSC) curve as set forth in the accompanying FIG. 6. Depending on the test conditions, the DSC curve of Amorphous Form of Compound I can be similar to or substantially similar to that as set forth in FIG. 6.

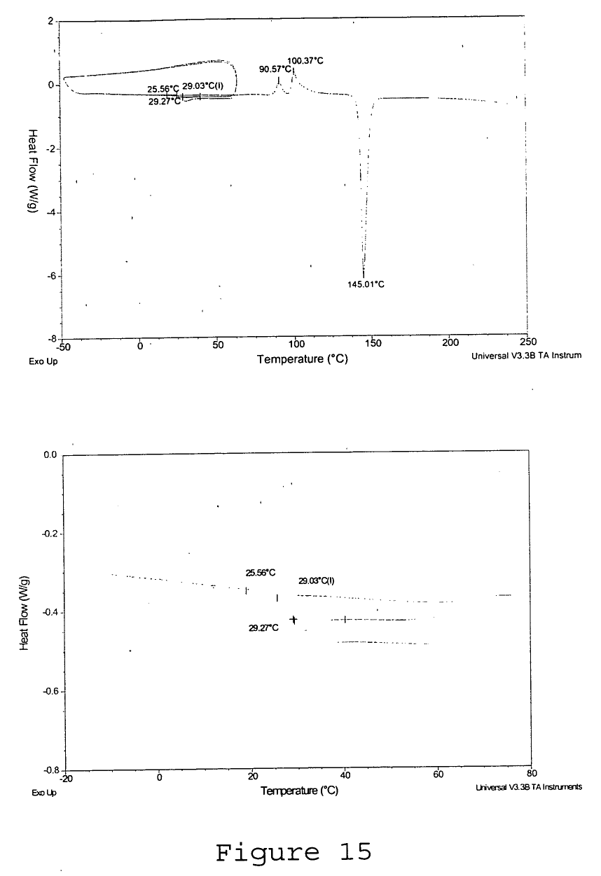

Additionally or alternatively, Amorphous Form of Compound I may also be characterized by the differential scanning calorimetric (DSC) curves similar or substantially similar to that set forth in the accompanying FIG. 15 as measured with thermal cycling using a TA Instruments differential scanning calorimeter 2920 or equivalent. These curves show a glass transition temperature for Amorphous Form of approximately 30° C. at temperature gradient of 20° C./min.

In addition, the TG curve shows a minimal weight loss up to 140° C., suggesting that the amorphous material is not solvated. The material displays two exothermic events at 85 and 97° C., which are likely crystallizations, followed by a melting endotherm at 144° C., suggesting that upon heating, Amorphous Form crystallizes to Form II.

The slurry interconversion experiments described in Tables 9 and 10 reveal that either Form II or Form III can convert to Form I in a few hours to several days upon stirring as a slurry in an organic solvent. However, the converse is not true; i.e., Form I does not convert under these same conditions to either Form II or Form III, but rather remains unchanged. These results indicate that Form I is more stable than either Form II or III at 25° C. However, the DSC study mentioned above indicated that Form II is more stable than Form I at temperatures near 140° C. All of these data taken together suggest that Forms I and II are related enantiotropically (Buerger, A.; Ramberger, R. “On the polymorphism of pharmaceuticals and other molecular crystals. I.” Mikrochim. Acta [Wein], 1979, II, 259-271.), with Form I being the more stable form at lower temperatures and Form II being more stable at higher temperatures (>100° C.). Thermal stress experiments described in Table 7 indicated that Forms I, II and III are all relatively stable to heat stress. All three polymorphs are also non-hygroscopic, as indicated by the data in Table 8.

Single crystals of Forms I and II were prepared for single crystal X-ray diffraction in order to determine which imine stereoisomer is present in each of these two crystalline forms. It was found that the crystals of both Forms I and II contain molecules of the E isomer of Compound I. Forms I and II are therefore polymorphs of each other because they represent two different crystal forms of the same stereoisomer.

Data presented in this invention further indicate that Form I may be one of the desirable forms for pharmaceutical development, because of its greater stability at ambient temperature, and its lack of salvation or hygroscopicity, and its crystallinity.

Pharmaceutical compositions of Form I, Form II, Form III or Amorphous Form of Compound I can be made by admixing a therapeutically effective amount of the compound of this invention with a pharmaceutically acceptable carrier.

A solid carrier can include one or more substances which may also act as endogenous carriers (e.g. nutrient or micronutrient carriers), flavoring agents, lubricants, suspending agents, fillers, glidants, compression aids, binders or tablet-disintegrating agents. In powders, the carrier is a finely divided solid which is in admixture with the finely divided Form I, Form II, Form III or Amorphous Form of Compound I. In tablets, Form I, Form II, Form III or Amorphous Form of Compound I is mixed with a carrier having the necessary compression properties in suitable proportions and compacted in the shape and size desired. The powders and tablets preferably contain up to 99% of Form I, Form II, Form III or Amorphous Form of Compound I. Suitable solid carriers include, for example, calcium phosphate, magnesium stearate, talc, sugars, lactose, dextrin, starch, gelatin, cellulose, low melting waxes and ion exchange resins.

Liquid carriers are used in preparing suspensions, emulsions, syrups, elixirs and pressurized compositions. Such suspensions, emulsions, syrups, elixirs and pressurized compositions may be prepared from Form I, Form II, Form III or Amorphous Form of Compound I either prior to packaging and distribution to patients or at the time of administration. Form I, Form II, Form III or Amorphous Form of Compound I can be dissolved or suspended in a pharmaceutically acceptable liquid carrier such as an aqueous solution, an organic solvent, a pharmaceutically acceptable oil or fat or a mixture of any of these. The liquid carrier can contain other suitable pharmaceutical additives such as emulsifiers, buffers, preservatives, sweeteners, flavoring agents, suspending agents, thickening agents, coloring agents, viscosity regulators, stabilizers or osmoregulators. Suitable examples of liquid carriers for oral and parenteral administration may include alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate or isopropyl myristate. Sterile liquid carriers are useful in sterile liquid form compositions for parenteral administration. The liquid carrier for pressurized compositions can be a halogenated hydrocarbon or other pharmaceutically acceptable propellent.

Liquid pharmaceutical compositions which are sterile solutions or suspensions can be utilized by for example, intramuscular, intrathecal, epidural, intraperitoneal or subcutaneous injection. Sterile solutions can also be administered intravenously. Form I, Form II, Form III or Amorphous Form of Compound I may be prepared as a sterile solid composition which may be dissolved or suspended at the time of administration using appropriate sterile injectable medium. Carriers are intended to include necessary and inert binders, suspending agents, lubricants, flavorants, sweeteners, preservatives, dyes, and coatings. The compound can also be administered orally in the form of a solution or suspension containing other solutes or suspending agents, bile salts, acacia, gelatin, sorbitan monoleate, polysorbate 80 (oleate esters of sorbitol and its anhydrides copolymerized with ethylene oxide) and the like.

Form I, II, III and Amorphous Form of Compound I can be administered orally or parenterally. Compositions suitable for oral administration may include solid forms, such as pills, capsules, granules, tablets, and powders, and liquid forms, such as solutions, syrups, elixirs, and suspensions. Forms useful for parenteral administration include sterile solutions, emulsions, and suspensions.

It should be noted that in the subject application a “therapeutically effective amount” is any amount of Compound I which, when administered to a subject suffering from a disease against which Compound I is effective, causes reduction, remission, or regression of the disease. In a subject application, a “subject” is a vertebrate, a mammal or a human.

Optimal dosages to be administered may be determined by those skilled in the art, and will vary with the particular solid Form of Compound I in use, the strength of the preparation, the mode of administration, and the advancement of the disease condition. Additional factors depending on the particular subject being treated will result in a need to adjust dosages, including subject age, weight, gender, diet, and time of administration.

In one embodiment, the amount of the compound is from about 0.01 mg to about 800 mg. In another embodiment, the amount of the compound is from about 0.01 mg to about 500 mg. In yet another embodiment, the amount of the compound is from about 0.1 mg to about 250 mg. In another embodiment, the amount of the compound is from about 0.1 mg to about 60 mg. In yet another embodiment, the amount of the compound is from about 1 mg to about 20 mg.

Preparation of Compound I

The present invention further provides a process for the preparation of 1-phenyl-3-[[3-(trifluoromethyl)phenyl]imino]-1H-indol-2-one (Compound I) as described herein. The process produces the desired compound in high yield and in the preferred polymorphic form (Form I) in a single pot process.

The process of the invention may be presented by the following scheme:

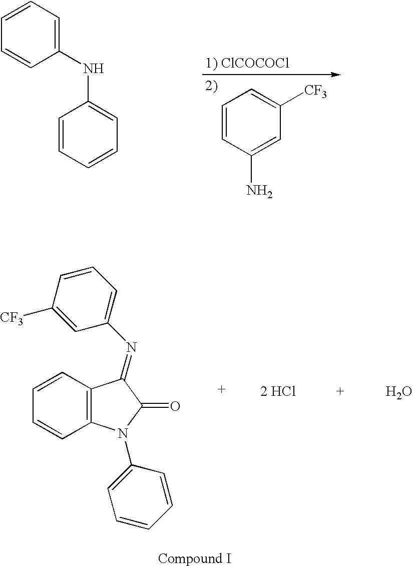

Compound I is produced in one pot from the reaction between diphenylamine, oxalyl chloride and 3-(trifluoromethyl)aniline in a suitable solvent such as toluene. A toluene solution of diphenylamine is added slowly to a toluene solution of oxalyl chloride while maintaining the reaction temperature below 30° C. The mixture is then heated to 60° C. for one hour.

Alternatively, the reaction temperature can be maintained at approximately 50 to 60° C. during the addition of diphenylamine. The resulting N,N-diphenyl oxamic chloride ((diphenylamino)oxoacetyl chloride) that is presumably formed can then be cyclized to form 1-phenylisatin in the presence of a Lewis acid or at high temperatures (100° C.-130° C.). After the cyclization is complete, a toluene solution containing 3-(trifluoromethyl)aniline is added. The water that is generated during the reaction is removed and collected via azeotropic distillation into a Dean-Stark apparatus. To aid in the crystallization and isolation of the product (Compound I) a suitable solvent such as heptane may be added to the hot reaction vessel. Upon cooling, the desired compound crystallizes from the reaction vessel to afford compound I in high purity and yield. The Form I polymorph of Compound I may be obtained in high purity if after cooling, the reaction slurry is allowed to stir for an appropriate amount of time before collection of the solids by filtration.

It should be noted that the term of “suitable solvent” shall include, but not be limited to, toluene and other solvents listed in this application. Upon following the teachings of this application, a person skilled in the art should be able to identify other solvents which may be applicable.

In particular, this present invention provides for the formation of each of Form I, Form II, Form III and Amorphous Form of Compound I or the formation of each of these forms of substantial purity.

Form I can be obtained in a straightforward fashion by stirring a slurry of any solid form of Compound I, and particularly Form II or III, in an organic solvent in which Compound I is partially soluble, for several hours until the material converts completely to Form I. Formation of Form I can be analyzed through DSC or, preferably, XRPD analysis. In one embodiment of the invention, a slurry of solid Compound I in an organic solvent is generated by cooling a hot, saturated solution of Compound I in an organic solvent, allowing crystals to form, then stirring the resulting slurry for several hours or a few days until the solid material converts substantially or completely to Form I. In a preferred embodiment, the hot, saturated solution of Compound I is generated in situ in the one-pot synthetic process described above. Form I can also be prepared by slurrying for several hours Compound I, Form II in a saturated methanol solution or in an appropriate solvent.

This invention also includes a method for the reproducible and controlled generation of substantially pure Form II, which comprises heating a mixture of Compound I in an organic solvent to a temperature at or near the boiling point of the solvent until the compound completely dissolves. The mixture is then allowed to cool until crystals just begin to form, then cooled rapidly to room temperature and filtered immediately, before interconversion to Form I can occur.

Compound I, Form II can also be prepared by crystallization from the slow or fast evaporation, rotary evaporation, or slow cooling of solutions of Compound I using a variety of solvents, including but not limited to acetone, dichloromethane, ethanol, methanol, tetrahydrofuran, or toluene.

Form III can be prepared from lyophilizing a solution of Compound I in t-butanol, or from a cold antisolvent crystallization using dichloromethane/hexanes, ethyl acetate/hexanes, or isopropylacetate/heptane solvent systems.

The following example is provided to illustrate the invention without, however, limiting the invention to the particulars of the example.

### EXAMPLE 1

To a stirring solution containing toluene (242 mL) and oxalyl chloride (172.8 g, 1.36 mol), under a flow of nitrogen, is added dropwise a solution of diphenylamine (212.64 g, 1.26 mol) in toluene (351 mL). During the addition, the reaction temperature is kept below 10° C. with the aid of an ice-bath. After addition is complete, the reaction mixture is heated at 60° C. for one hour during which time HCl gas evolution ceases and a clear light brown solution is obtained. The reaction solution is then distilled (˜175 mm Hg; Pot Temp 72° C.) to remove the excess oxalyl chloride. Approximately 300 mL of distillate is collected. The dark red-brown solution is then heated at 120° C. for approximately 19 h. The reaction solution is cooled to 95° C. and a Dean-Stark apparatus is fitted to the flask. A solution of 3-(trifluoromethyl)aniline (263 g, 1.63 mol) dissolved in toluene (529 mL) is then added dropwise over a period of approximately one hour while maintaining the reaction temperature at 95-100° C. After addition is complete, the dropping funnel is rinsed with toluene (83 mL) and the liquid is added to the reaction solution. The reaction is heated at 100-118° C. until the water that is collected is close to the theoretical amount (22.6 mL). The reaction mixture is cooled to 90° C. and approximately 440 mL solvent is distilled under vacuum (91-95° C., ˜380 mm Hg). Heptane (650 mL) is then added slowly while maintaining a pot temperature of 90-95° C. and the solution is then allowed to cool to room temperature while stirring for overnight. To the resulting thick pasty orange solid, heptane (400 mL) is added, the mixture is stirred vigorously and the solid is collected by filtration. The solid is placed in a vacuum oven to dry at 40° C. (401.1 g, 87% yield).

Compound I may be recrystallized by combining 3 mL toluene per gram of Compound I. The mixture is heated to 60° C.-70° C. until most of the solids dissolve. The solution is hot filtered and allowed to cool to room temperature, while stirring, for overnight. The orange solids are collected by vacuum filtration and dried in a vacuum oven at 40° C. (80% recovery yield). Analysis by DSC and XRPD shows that the compound is pure Form I.

### EXAMPLE 2

A sample of approximately 112 mg of Compound I, Form II material was suspended in 1 mL of a saturated methanol solution of Compound I and agitated for at least 4 hours. The resulting solids were filtered and air-dried to yield Form I of Compound I.

### EXAMPLE 3

A sample of 102.5 mg Compound I was dissolved in 0.2 mL tetrahydrofuran. The solution was filtered through a 0.2 μm nylon filter into a vial which was left open to quickly evaporate the solvent. The resulting solids were vacuum filtered and air-dried to yield Form II of Compound I.

### EXAMPLE 4

A sample of 33.4 mg of Compound I was dissolved in 0.1 mL dichloromethane and the solution heated to 60° C. The solution was filtered through a warm 0.2 μm nylon filter into 1.5 mL of hexanes that had been placed in a dry ice/acetone bath. After cooling the solution for 20 minutes, solids formed, which were collected by vacuum filtration and air-dried to yield Form III of Compound I.

### EXAMPLE 5

A sample of Compound I was melted and quenched to room temperature by removing the heat source and placing the melt on a cool surface to yield Amorphous Form of Compound I.

TABLE 1
Polymorph Screen of Compound I

| Solvent | Conditions^{a} | XRPD Results^{b} |
|---|---|---|
| acetone | FE | II |
| | SE | II |
| | SC (45° C.) | II |
| acetonitrile | FE | II (PO) |
| | SE | II |
| | SC (60° C.) | II |
| Dichloromethane | FE | II (PO) + pk@5.5, 17.5° 2θ |
| (DCM) | SE | II (PO) + pk@5.5° 2 θ |
| | SC (45° C.) | — |
| | rotovap | II |
| | rotovap | II |
| Diethyl ether | FE | I (PO) |
| | SE | II (PO) |
| | SC (45° C.) | III + II (PO) |
| | rotovap | II (SS) |
| N,N-dimethyl- | FE | II (PO) |
| formamide (DMF) | SE | PO |
| ethanol | FE | II (PO) |
| | SE | II (PO) |
| | SC (60° C.) | II (PO) |
| | fast cool | II (PO) |
| ethyl acetate | FE | II |
| | FE | II (PO) |
| ethyl acetate | SE | II |
| | SC (60° C.) | I |
| hexanes | slurry | I |
| isopropanol | FE | II (PO) |
| | SE | II |
| | SC (60° C.) | II (PO) |
| | fast cool | II (PO) |
| methanol | FE | II (PO) |
| | SE | I |
| | SC (60° C.) | II (PO) |
| | fast cool | II |
| Toluene | FE | II |
| | SE | II (PO) |
| | SC (60° C.) | I |
| tetrahydrofuran | FE | II |
| (THF) | FE | II |
| | FE | II |
| | FE | II |
| THF | FE | II |
| | SE | II |
| | SC (45° C.) | — |
| | rotovap | II |
| Water | slurry | I |
| None | Melt/quench | amorphous |

^{a}FE = fast evaporation; SE = slow evaporation; SC = slow cool, rotovap = rotary evaporator
^{b}PO = preferred orientation; SS = small sample; pk = peak

TABLE 2
Cold Precipitation Crystallizations

| Solvent | Anti-solvent | XRPD Results^{a} |
|---|---|---|
| acetone | water | II |
| acetonitrile | water | II |
| dichloromethane | hexanes | III |
| | | III |
| | | III |
| | | III + II (min) |
| diethyl ether | hexanes | I (PO) |
| dimethylformamide | water | II |
| ethanol | hexanes | II (SS) |
| ethyl acetate | hexanes | III |
| | | III |
| isopropanol | water | II |
| methanol | water | II |
| tetrahydrofuran | hexanes | SS |

^{a}PO = preferred orientation; SS = small sample; min = minor

TABLE 3
Vapor Diffusion Experiments

| Solvent | Antisolvent | XRPD Results^{a} |
|---|---|---|
| acetone | water | II |
| acetonitrile | water | II |
| dichloromethane | hexanes | — |
| diethyl ether | hexanes | II (PO) |
| N,N-dimethyl-formamide | water | II (PO) |
| ethanol | hexanes | — |
| ethyl acetate | hexanes | — |
| isopropanol | water | I (PO) |
| methanol | water | II (PO) |
| tetrahydrofuran | hexanes | — |
| toluene | water | — |

^{a}PO = preferred orientation

TABLE 4
XRPD Peak locations and Intensities of all Diffraction Lines with
Relative Intensities Greater than 10% for Compound I, Form I

| 2θ | Relative Intensity (>10%)^{a} |
|---|---|
| 6.3 | 100 |
| 12.7 | 16 |
| 19.0 | 83 |
| 22.0 | 14 |
| 24.9 | 14 |
| 25.5 | 33 |
| 38.6 | 12 |

^{a}Peaks listed are >10% relative intensity. 2θ values are listed ±0.1° as listed in the U.S. Patent No. <941>. Due to the presence of one large peak (due to preferred orientation) threshold was lowered to account for the relevant peaks.

TABLE 5
XRPD Peak Locations and Intensities of all Diffraction Lines with
Relative Intensities Greater than 10% for Compound I, Form II

| 2θ | Relative Intensity (>10%)^{a} |
|---|---|
| 6.0 | 100 |
| 12.1 | 31 |
| 13.2 | 26 |
| 13.7 | 12 |
| 16.0 | 45 |
| 16.3 | 60 |
| 16.7 | 33 |
| 18.2 | 77 |
| 19.0 | 50 |
| 19.9 | 38 |
| 20.8 | 28 |
| 21.7 | 38 |
| 22.0 | 64 |
| 24.5 | 49 |
| 25.1 | 23 |
| 25.6 | 63 |
| 26.8 | 47 |
| 29.1 | 14 |
| 30.2 | 16 |
| 33.1 | 12 |

^{a}Peaks listed are >10% relative intensity. 2θ values are listed ±0.1° as listed in the U.S. Patent No. <941>.

TABLE 6
XRPD Peak Locations and Intensities of all Diffraction Lines with
Relative Intensities Greater than 10% for Compound I, Form III

| 2θ | Relative Intensity (>10%)^{a} |
|---|---|
| 6.1 | 73 |
| 12.2 | 27 |
| 15.5 | 15 |
| 16.0 | 51 |
| 16.4 | 34 |
| 17.2 | 45 |
| 18.4 | 28 |
| 19.3 | 24 |
| 20.3 | 41 |
| 21.2 | 100 |
| 21.6 | 22 |
| 22.3 | 12 |
| 23.1 | 11 |
| 24.4 | 12 |
| 25.0 | 21 |
| 25.7 | 52 |
| 26.5 | 45 |
| 27.8 | 16 |

^{a}Peaks listed are >10% relative intensity. 2θ values are listed ±0.1° as listed in the U.S. Patent No. <941>.

TABLE 7
Stress Studies of Compound I

| Form | Conditions | Result^{a} |
|---|---|---|
| amorphous | 80 C./4 hours | II |
| amorphous | 120 C., 15 min | II |
| amorphous | 60 C./17 hours | III (LC) |
| I | 60 C., 4 days | I |
| I | 70 C., 4 days | I |
| I | 80 C., 4 days | I |
| II | 60 C., 4 days | II |
| II | 70 C., 4 days | II |
| II | 80 C., 4 days | II (PO) + peak@5° 2θ |
| III | 60 C., 4 days | III (SS) |
| III | 70 C., 4 days | III (SS) |
| III | 80 C., 4 days | III (SS) |
| III | 135 C., 5 min | III |

^{a}PO = preferred orientation; SS = small sample; LC = low crystallinity

TABLE 8
Summary of Moisture Sorption/Desorption
Data for Compound I Forms

| | | XRPD |
|---|---|---|
| Form | Moisture Balance Results | Results |
| I | <0.1% weight loss at 5% RH, | I |
| | <0.1% total weight gain at | |
| | 95% RH | |
| II | <0.1% weight loss at 5% RH, | II |
| | <0.1% total weight gain at | |
| | 95% RH | |
| III | <0.1% weight loss at 5% RH, | III |
| | <0.1% total weight gain at | |
| | 95% RH | |
| amorphous | <0.1% weight gain at 5% RH, | amorphous |
| | 0.3% total weight gain at | |
| | 95% RH | |

a. XRPD results on solid after moisture balance run

TABLE 9
Slurry Experiments on Compound I Crystalline Forms

| Experi- | | | | |
|---|---|---|---|---|
| ment | Form | Solvent | Slurry Conditions | XRPD Results^{a} |
| 1 | I | MeOH | ambient, 11 d | I (PO) |
| 2 | II | MeOH | ambient, 11 d | I (PO) |
| 3 | II | MeOH | 25° C., 1 hour | II + I (minor) |
| 3 | II | MeOH | 25° C., 4 hours | I |
| 3 | II | MeOH | 25° C., 7 hours | I |
| 3 | II | MeOH | 25° C., 12.5 hours | I |
| 4 | II | MeOH | 25° C., 20 min | II |
| 4 | II | MeOH | 25° C., 40 min | II |
| 4 | II | MeOH | 25° C., 1 hour | I + II |
| 4 | II | MeOH | 25° C., 1 hour 20 min | I + II |
| 4 | II | MeOH | 25° C., 1 hour 40 min | I + II (minor) |
| 4 | II | MeOH | 25° C., 2 hours | I |

TABLE 10
Interconversion Experiments of Compound I

| | | Slurry Time | XRPD |
|---|---|---|---|
| Forms | Solvent | (days) | Results^{a} |
| I/II | IPA | 11 | I |
| I/III | IPA | 11 | I |
| II/III | IPA | 11 | I |
| I/II | EtOH/water (4:1) | 13 | I |
| I/III | EtOH/water (4:1) | 13 | I |
| II/III | EtOH/water (4:1) | 13 | I (LC) |
| I/II | 1-butanol (52° C.) | 7 | I |
| I/II | 1-butanol (52° C.) | 7 | I |
| I/II | 1-butanol (61° C.) | 7 | I |
| I/II | 1-butanol (61° C.) | 7 | I |
| I/II | 1-butanol (70° C.) | 1 | I |
| I/II | 1-butanol (70° C.) | 1 | I |
| I/II | 1-butanol (80° C.) | 1 | I |
| I/II | 1-butanol (80° C.) | 1 | I |
| I/II | 1-octanol (100° C.) | 1 | I |
| I/II | 1-octanol (100° C.) | 1 | I |

^{a}LC = low crystallinity

TABLE 11
Comparison of Torsion Angles between Symmetry Independent
Molecules in Form I and II of Compound I

| Molecule | Torsion | Atoms^{a} | Torsion Angle (°)^{b} | |
|---|---|---|---|---|
| Form I, | τ₁ | C13-N131-C132- | 126.49 | (0.62) |
| molecule 1 | | C133 | | |
| Form I, | | C23-N231-C232- | 56.77 | (0.91) |
| molecule 2 | | C233 | | |
| Form II | | C3-N30-C31-C32 | −59.01 | (0.28) |
| Form I, | τ₂ | C12-N11-C111- | 123.88 | (0.64) |
| molecule 1 | | C112 | | |
| Form I, | | C22-N21-C211- | −125.58 | (0.65) |
| molecule 2 | | C212 | | |
| Form II | | C2-N1-C11-C12 | 125.54 | (0.21) |

^{a}Atoms numbering refers to FIGS. 7 and 11.
^{b}Numbers in parenthesis refer to standard deviation.

TABLE 12
Crystal Data and Data Collection
Parameters for COMPOUND I, Form I

| | |
|---|---|
| formula | C₂₁H₁₃F₃N₂O |
| formula weight | 366.35 |
| space group | P2₁/c (No. 14) |
| a, Å | 7.2704 (2) |
| b, Å | 16.9251 (5) |
| c, Å | 27.8017 (11) |
| β, deg | 92.6340 (10) |
| V, Å³ | 3417.4 (3) |
| Z | 8 |
| d_{calc,} g cm⁻³ | 1.424 |
| crystal dimensions, mm | 0.35 × 0.30 × 0.05 |
| temperature, K | 150. |
| radiation (wavelength) | MO K_{α}(0.71073Å) |
| monochromator | graphite |
| linear abs coef, mm⁻¹ | 0.105 |
| absorption correction applied | empirical^{a} |
| transmission factors: min, max | 0.65, 0.99 |
| diffractometer | Nonius KappaCCD |
| h, k, l range | −8 to 8 |
| | −20 to 20 |
| | −33 to 33 |
| 2θ range, deg | 5.02-51.51 |
| mosaicity, deg | 0.83 |
| programs used | SHELXL-97 |
| F₀₀₀ | 1504.0 |
| weighting | |

1/[σ²(Fₒ²) + (0.1142P)² + 11.2475P] where P = (Fₒ² + 2F_{c}²)/3

| | |
|---|---|
| data collected | 20535 |
| unique data | 6479 |
| Rᵢₙₜ | 0.165 |
| data used in refinement | 6457 |
| cutoff used in R-factor calculations | Fₒ² > 2.0σ(Fₒ²) |
| data with I > 2.0σ(I) | 4863 |
| refined extinction coef | 0.0340 |
| number of variables | 488 |
| largest shift/esd in final cycle | 0.05 |
| R(Fₒ) | 0.142 |
| R_{w}(Fₒ²) | 0.307 |
| goodness of fit | 1.183 |

^{a}Otwinowski Z. & Minor, W. Methods Enzymol., 1996, 276, 307.

TABLE 13
Crystal Data and Data Collection Parameters
for COMPOUND I, Form II

| | |
|---|---|
| formula | C₂₁H₁₃F₃N₂O |
| formula weight | 366.35 |
| space group | P2₁/c (No. 14) |
| a, Å | 14.7527(2) |
| b, Å | 16.9524(6) |
| c, Å | 7.0196(5) |
| β, deg | 100.8840(19) |
| V, Å³ | 1724.0(2) |
| Z | 4 |
| d_{calc}, g cm⁻³ | 1.411 |
| crystal dimensions, mm | 0.38 × 0.33 × 0.13 |
| temperature, K | 150. |
| radiation (wavelength) | Mo Kα(0.71073Å) |
| monochromator | graphite |
| linear abs coef, mm⁻¹ | 0.104 |
| absorption correction applied | empirical^{a} |
| transmission factors: min, max | 0.89, 0.99 |
| diffractometer | Nonius KappaCCD |
| h, k, l range | −19 to 19 |
| | −22 to 15 |
| | −9 to 9 |
| 2θ range, deg | 10.06-54.99 |
| mosaicity, deg | 0.32 |
| programs used | SHELXL-97 |
| F₀₀₀ | 752.0 |
| weighting | |

1/[σ²(Fₒ²) + (0.0851P)² + 0.0000P] where P = (Fₒ² + 2F_{c}²)/3

| | |
|---|---|
| data collected | 13366 |
| unique data | 3920 |
| Rᵢₙₜ | 0.054 |
| data used in refinement | 2995 |
| cutoff used in R-factor calculations | Fₒ² > 2.0σ(Fₒ²) |
| data with I > 2.0σ(I) | 2061 |
| number of variables | 262 |
| largest shift/esd in final cycle | 0.00 |
| R(Fₒ) | 0.051 |
| R_{w}(Fₒ²) | 0.129 |
| goodness of fit | 1.004 |

^{a}Otwinowski Z. & Minor, W. Methods Enzymol., 1996, 276, 307.

TABLE 14
Infrared Peaks Unique for Form I, II and III.

| Form | Unique Peaks (cm⁻¹)^{a} |
|---|---|
| I | 3462, 3285, 3106, 2770, 2752, |
| | 1991, 1882, 1747, 1696, 1656, |
| | 1651, 1332, 1253, 557S |
| II | 3468, 3291, 3111, 3076, 2962, |
| | 2807, 1995, 1967, 1896, 1751, |
| | 1699, 1659, 1340, 1326, 1261, |
| | 1230, 1182, |
| III | 3450, 3297, 3058, 3101, 2810, |
| | 1982, 1972, 1930, 1888, 1820, |
| | 1742, 1691, 1663, 1336, 1288, |
| | 1250, 1196, 975, 873, |

^{a}for data collected at a resolution of 4 cm⁻¹

TABLE 15
Raman Peaks Unique for Form I, II and III.

| Form | Unique Peaks (cm⁻¹) |
|---|---|
| I | 1739, 1653 |
| II | 1742, 1658 |
| III | 1734, 1662, 1333, 1178 |

a. for data collected at a resolution of 4 cm − 1

1. A crystalline form I of compound I having an X-ray powder diffraction pattern comprising the following 2θ value measured using CuKα radiation: 6.3.

2. A crystalline Form I of Compound I having an X-ray powder diffraction pattern comprising the following 2θ values measured using CuKα radiation: 6.3, 19.0 and 25.5.

3. A crystalline Form I of Compound I of claim 2 diffraction pattern further comprising the following 2θ values: 12.7, 22.0, 24.9 and 38.6.

4. A crystalline Form I of Compound I having an X-ray powder diffraction pattern substantially similar to that set forth in FIG. 1a as measured using CuKα radiation.

5. A crystalline Form I of Compound I having differential scanning calorimetric curves substantially similar to those set forth in FIG. 3.

6. A crystalline Form I of Compound I having differential scanning calorimetric curves comprising one endotherm at approximately 141° C. and one endotherm at approximately 143° C., as measured at a ramp rate of 1° C./min.

7. A crystalline Form I of Compound I having a Fourier transform infrared pattern comprising at least one of the following infrared peaks: 3462, 3285, 3106, 2770, 2752, 1991, 1882, 1747, 1696, 656, 1651, 1332, 1253 and 557.

8. A crystalline Form I of Compound having a Raman peak pattern comprising at least one of the following peaks: 1739 and 1653, as measured using a spectrometer.

9. A pharmaceutical composition useful for treatment of a human disease comprising crystalline Form I of Compound I and a pharmaceutically acceptable carrier.

10. The composition of claim 9, wherein a substantial percentage of Compound I is present as Form I.

11. The composition of claim 9, wherein at least 99.9% of Compound I is present as Form I.

12. The composition of claim 9, wherein at least 98% of Compound I is present as crystalline Form I.

13. The composition of claim 9, wherein at least 95% of Compound I is present as crystalline Form I.

14. The composition of claim 9, wherein at least 90% of Compound I is present as crystalline Form I.

15. The composition of claim 9, wherein at least 85% of Compound I is present as crystalline Form I.

16. The composition of claim 9, wherein at least 80% of Compound I is present as crystalline Form I.

17. The composition of claim 9, wherein the disease is depression or anxiety.

18. A process for preparation of a pharmaceutical composition, comprising admixing Form I of Compound I with a pharmaceutically acceptable carrier.

19. The process of claim 18, further comprising obtaining Form I of Compound I of substantial purity.

20. A method for treatment of a human disease, wherein the method comprises administering to a human subject suffering such disease a therapeutically effective amount of crystalline Form I of Compound I.

21. The method of claim 20, wherein the disease is a CNS disorder.

22. The method of claim 20, wherein the disease is anxiety or depression.

23. A process of preparing of crystalline Form I of Compound I, comprising stirring a slurry of Compound I in a solvent for a period of time of no less than one hour.

24. The process of claim 23, wherein the solvent is selected from a group consisting of toluene, heptane, meta-xylene, ortho-xylene, para-xylene, isopropyl acetate, methanol, ethanol, 1-butanol, 1-octanol.

25. A crystalline Form III of Compound I having an X-ray powder diffraction pattern comprising at least one of the following 2θ values measured using CuKα radiation: 6.1 and 21.2.

26-93. (canceled)
